(19) United States
(12) Patent Application Publication
(10) Pub. No.: US 2005/0256373 A1
(43) Pub. Date: Nov. 17, 2005

(54) Disposable set for use with an endoscope

(75) Inventors: Yakov Bar-Or, Haifa (IL); Amram Aizenfeld, Kibbutz Ramot Menashe (IL); Golan Salman, Tirat Hacarmel (IL)

Correspondence Address: BRUCE E. LILLING;LILLING & LILLING P.C., P.O. BOX 560, GOLDEN BRIDGE, NY, 10526, US

(73) Assignee: Sightline Technologies Ltd., Haifa (IL)

(21) Appl. No.: 11/121,692

(22) Filed: May 4, 2005

Related U.S. Application Data
(60) Provisional application No. 60/570,608, filed on May 13, 2004.

(51) Int. Cl. A61B 1/00

(52) U.S. Cl. 600/114; 600/121

(57) ABSTRACT

A disposable set for use with an endoscope is described. The set comprises a dispenser provided with a longitudinal transit passage adapted for passing the endoscope therealong. The dispenser stores a disposable multilumen tubing fitted at a distal end thereof with a cap that is detachably connectable to an optical head of the endoscope. The dispenser is fitted with a protective sleeve adapted to cover the endoscope during the endoscopic procedure. The dispenser is adapted for receiving a lubricant and distributing thereof within the transit passage.

### FIELD OF THE INVENTION

The present invention relates generally to the field of endoscopy and specifically to endoscopic apparatus used for colonoscopic procedures during which a flexible tube is inserted into the rectum and colon for examination of the colon interior for abnormalities. More particularly, the present invention refers to a disposable set for use with endoscopes and having a disposable sleeve covering the tube and which upon inflation enables advancement of the colonoscope within the colon.

### BACKGROUND OF THE INVENTION

There are known endoscopes employing inflatable flexible sleeves for propulsion of an endoscope within the colon.

Voloshin (U.S. Pat. No. 6,485,409) discloses an endoscope, which comprises an endoscopic probe, a bending section for directing the probe within the colon (steering unit), an insertion tube and a flexible covering sleeve or a sheath, which is coupled proximally to the probe. The bending section of the endoscope is located behind the probe. The sleeve is attached to the endoscope in such a manner that its folded section is retained between a cap and an internal spindle, which are located between the insertion tube and the steering head. When inflated, the folded section unfolds over a flange of the internal spindle and an inner portion of the sleeve is pulled in a distal direction.

In PCT/IL03/00661 is described endoscope employing a flexible inflatable sleeve, which before inflation is retained within a dispenser. The dispenser employed in this endoscope has entry and exit ports defining a transit passage through which the endoscope may pass. The dispenser is adapted to capture the sleeve as the endoscope is retracted through the transit passage in a proximal direction. In another embodiment the dispenser includes an external sleeve fixed to the dispenser and this external sleeve is adapted to extend from the dispenser when the endoscope is retracted so that the external sleeve covers the flexible sleeve. By virtue of this provision any contamination on the flexible sleeve remains within the external sleeve and does not contact the endoscope or any other objects or areas outside the patient's body. After the endoscope has been evacuated entirely from the flexible sleeve, the dispenser together with the external sleeve and the flexible sleeve is discarded.

It is mentioned in the above reference that the endoscope is provided with an internal working channel, which is also known as multilumen tubing, since it is usually fitted with several passages or lumens as required for irrigation, suction and for passing endoscopic tools therethrough. The multilumen tubing extends along a guiding channel provided within the insertion tube.

However neither construction of the dispenser nor construction of the arrangement associated with the external sleeve and the internal sleeve is disclosed in the above reference.

It should be also noted that during functioning of the dispenser unavoidable friction between the insertion tube and the interior of the transit passage of the dispenser takes place, rendering operation of the endoscope inconvenient and less efficient. No measures are described in the above reference, which could lubricate the transit passage and at least partially reduce the friction.

### SUMMARY OF THE INVENTION

The object of the present invention is to provide a new and improved flexible endoscope employing a disposable sleeve covering the endoscope during the endoscopic procedure. The endoscope of the present invention is provided with a sleeve-retaining dispenser of the kind described in the above reference, including arrangement for the external sleeve, and in this sense the dispenser of the present invention is functionally similar with the prior art dispenser. The dispenser of the present invention is fitted with an arrangement for distributing antifriction fluid material within the transit passage in order to reduce friction during displacement of the insertion tube along the dispenser. The endoscope of the present invention is also provided with an internal sleeve or multilumen tubing, which constitutes an integral body fitted with dedicated passages (lumens) extending therealong as required for functioning the endoscope.

Since the retaining dispenser as well as the multilumen tubing are discardable after use, the present invention concerns a disposable set for use with the endoscopic apparatus.

For a better understanding of the present invention as well of its benefits and advantages, reference will now be made to the following description of its embodiments taken in combination with the accompanying drawings.

### BRIEF DESCRIPTION OF THE DRAWINGS

FIG. 1 depicts a general view of endoscopic apparatus of the invention, preferably acolonoscopic apparatus and its main components.

FIG. 2 is a general view of a disposable set of the invention.

FIG. 3 is a general view of a disposable set of the invention, depicting when the distal end of the multilumen tubing is inserted within the dispenser and its proximal end is ready for insertion within the guide channel of the insertion tube.

FIG. 4 is an enlarged general view of a disposable set of the invention, depicting when the proximal end of the multilumen tubing is almost fully inserted within the insertion tube and is ready for insertion within the dispenser.

FIG. 5 is a general view of a disposable set of the disposable multilumen tubing outside the dispenser.

FIG. 6 is a cross-sectional view of the dispenser with the multilumen tubing inside of the dispenser.

FIG. 7 is an enlarged general view of a disposable set of the invention, showing the distal end of the endoscope protruding from the dispenser with an alignment member almost withdrawn from the cap of the multilumen tubing.

FIG. 8 is an enlarged general view of a disposable set of the invention, showing the distal end of the endoscope protruding from the dispenser.

FIG. 9 is a cross-sectional view of the dispenser with distal end of the endoscope protruding therefrom.

FIG. 10 is a cross-sectional view of the dispenser when the distal end of the endoscope is advanced through the transit passage.

FIGS. 11 and 12 are views, showing how the plunger member is manually pushed so as to distribute antifriction material within the transit passage of the dispenser.

FIG. 13 is a cross-sectional view of the dispenser with the plunger member in its most protracted position.

FIG. 14 is a general view of a disposable set of the invention, showing the dispenser with the distal end of the endoscope before commencing an endoscopic procedure.

FIG. 15 is a cross-sectional view of the dispenser during an endoscopic procedure.

FIG. 16 is a cross-sectional view of the dispenser at the beginning of deployment of the external sleeve over the flexible sleeve.

FIG. 17 is a cross-sectional view of the dispenser at the end of deployment of the external sleeve.

FIGS. 18 and 19 are enlarged general views of a disposable set of the invention, showing correspondingly the dispenser with the external sleeve at the beginning and at the end of deployment.

FIG. 20 is a cross-sectional view, showing an alternative embodiment of the set of the present invention.

### DETAILED DESCRIPTION OF THE INVENTION

With reference to FIG. 1 the endoscopic apparatus of the present invention is shown preferably as a colonoscopic apparatus 10 with its following main components. The apparatus comprises an endoscope having insertion tube 12 with its proximal section connected to an operation handle 14 and with its distal section 16 inserted in and protruding from a disposable dispenser 18.

It is seen also in FIG. 1 that a sleeve covers distal section 16 of the endoscope. That part of the sleeve, which is seen in FIG. 1, comprises a frontal non-inflatable portion 20 and a rear, folded portion 22. The frontal portion of the sleeve covers the distal section of the endoscope and its head. The frontal portion does not inflate when the endoscope advances within the colon. The rear portion covers the insertion tube and unfolds when air or another fluid medium is supplied to the sleeve. By virtue of this provision the feeding out sleeve propels the endoscope within the body passage. Explanation of this phenomenon can be found in the above referred-to references. The endoscope of the present invention is of similar type in the sense that it employs the same propelling mechanism, which is based on inflation of the flexible disposable sleeve coupled to the endoscope. It should be appreciated, however, that the endoscope of the present invention is not limited merely to colonoscopy. It can be employed in any other medical procedure requiring insertion of a probe in a body passage for inspection of its interior.

It is seen also in FIG. 1, that the handle is connected by appropriate umbilical duct 24 to a control unit 26 provided with a source of compressed air for inflating and venting the sleeve. Proximate to the control unit a flask 28 is provided, which is filled with water, to be supplied under pressure into the colon for irrigation. Water and air are supplied to the handle through dedicated tubes (not shown) attachable to the umbilical duct.

One should bear also in mind that within the insertion tube are provided various devices, which are necessary for proper functioning of the endoscope. These devices are known per se. Among such devices one can mention vertebrae and strings, which can be manipulated by the handle and a multilumen tubing with appropriate passages for supplying water, as required for irrigation or vacuum as required for suction. The multilumen tubing also is provided with a dedicated passage for introducing surgical instruments into the colon as might be required during the endoscopic procedure. Along the insertion tube might extend also a dedicated tube through which air is supplied as required for inflating the sleeve.

The multulumen tubing extends through the endoscope and through the handle to a connector means 30, which provides flow communication between lumens of the multilumen tubing and tubes passing through the umbilical duct tubes and supplying air and vacuum from the control unit and water from the flask.

In FIG. 2 a general view of the disposable set of the invention is shown, including disposable dispenser 18 and a disposable multilumen tubing 32, having its distal section inserted within the dispenser and its proximal section extending outside the dispenser. An end 34 of the proximal section is adapted for airtight connection to the connector means 30.

It is not shown in FIG. 2, but it should be appreciated that to the distal end of the multilumen tubing is attached to a cap having a window fitted with transparent closure. Through this window illuminating light can propagate and thus viewing optics of a camera head may observe the interior of a body passage when the endoscope is inserted therein.

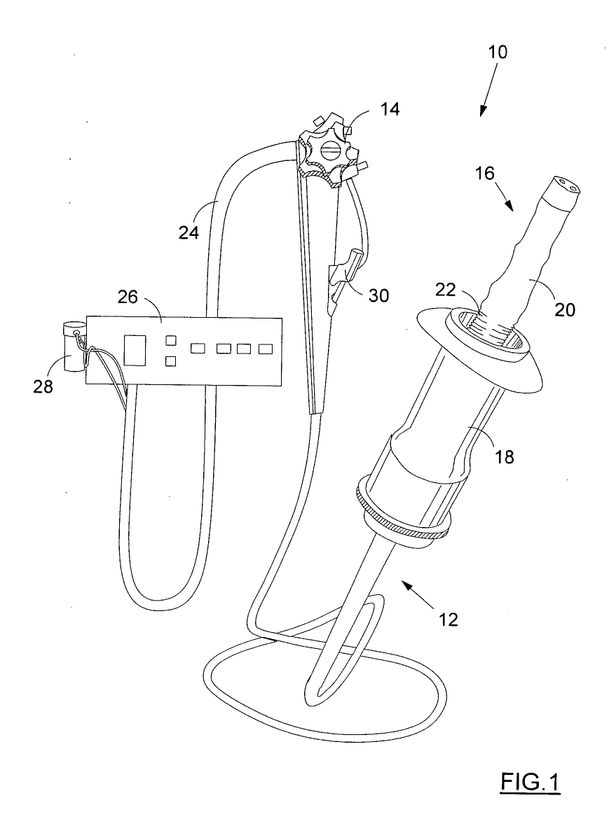

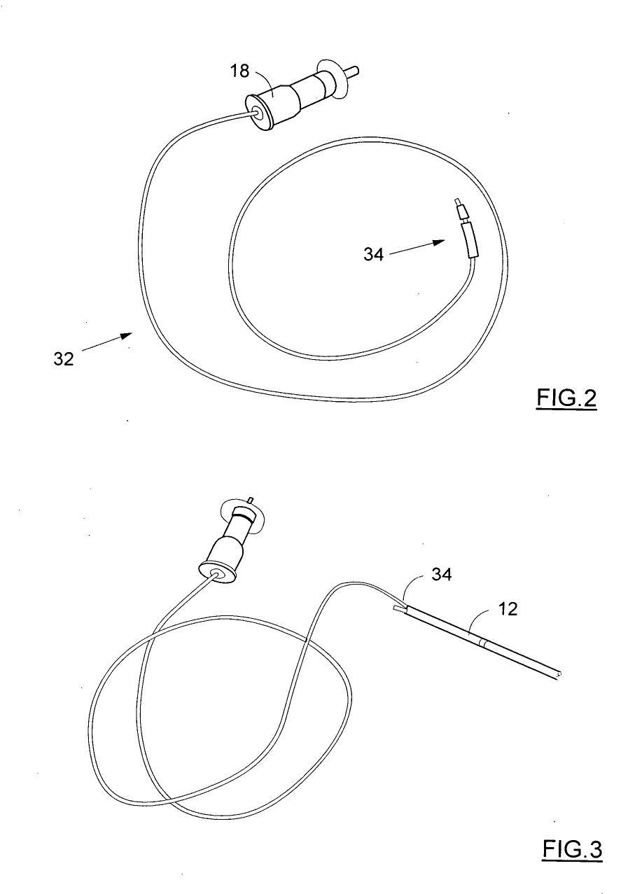

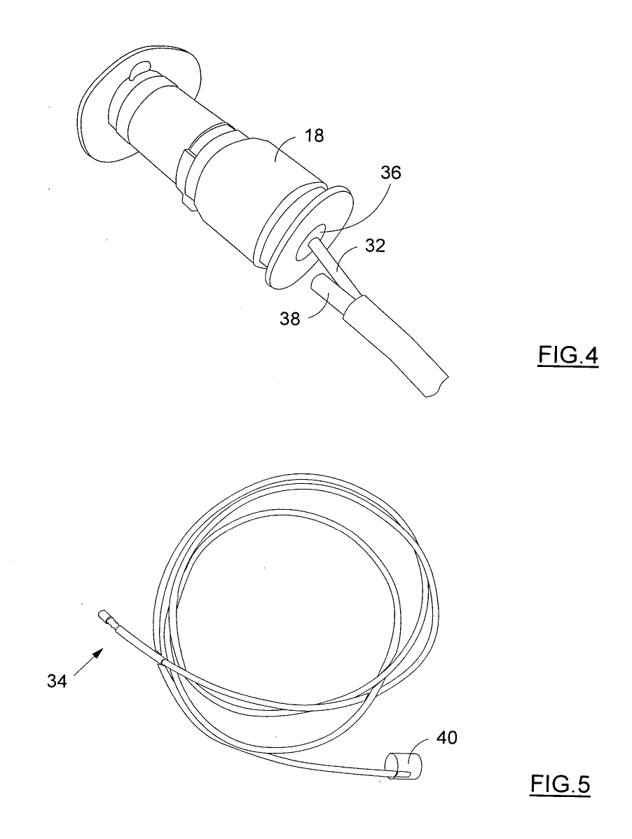

It should be also born in mind that in FIG. 2 the dispenser and the multilumen tubing are depicted during preparation for the endoscopic procedure. As shown in FIG. 3 and FIG. 4 during the preparation step the end 34 of the multilumen tubing is inserted within a guiding channel of the insertion tube 12. Then, distal section 16 of the endoscope is displaced along an arrow seen in FIG. 4 and is inserted into dispenser via an entry port 36. After that, the distal section of the endoscope is advanced along the dispenser until it protrudes therefrom. It is seen in FIG. 4, that distal section of the endoscope terminates by an optical head 38, which has a saddle-like cross-sectional configuration, thereby enabling the multilumen tubing to pass therethrough and through the guiding channel (not shown)

In FIG. 5 the multilumen tubing is shown outside the dispenser but it should be born in mind that during the preparation procedure the multilumen tubing is retained within the dispenser and constitutes a component of the disposable set.

It is also seen that to the distal section of the multilumen tubing is attached a cap 40. It is not shown specifically but should be appreciated that the cap has an access opening through which the interior of the body passage is accessible during the endoscopic procedure. The multilumen tubing is attached to the cap in such a manner that the access opening is in alignment with the passages of the multilumen tubing.

The dispenser, the multilumen tubing and the tubes passing along the umbilical duct are manufactured from plastic material; they are cheap, disposable items, which are discarded at the end of the endoscopic procedure after extracting the endoscope from the body passage. By virtue of this provision preparation of the endoscopic apparatus to the new endoscopic procedure is simple, convenient and fast. Furthermore, by virtue of the components of the disposable set, contamination from within the body passage is prevented.

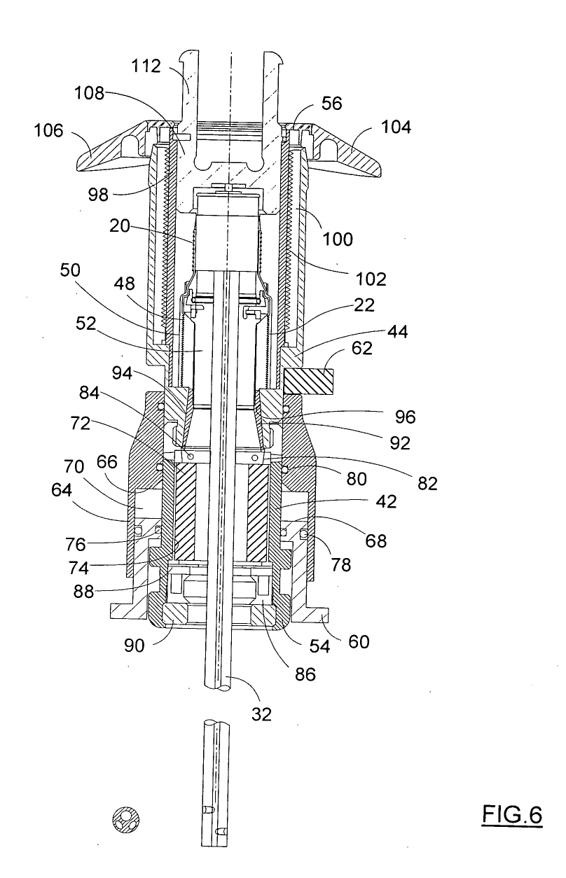

Referring now to FIG. 6 it shows a cross-section of the disposable set, including the dispenser, which retains inside the multilumen tubing 32. The distal section of the endoscope has not been inserted in the dispenser yet.

The disposable dispenser of the present invention is an assembly, which comprises the following main components: a rear housing 42, a frontal housing 44 and connecting therebetween an intermediate bushing 46. It is seen that the multilumen tubing 32 passes along the dispenser and that the dispenser retains a flexible, protective sleeve 48.

During the endoscopic procedure this sleeve upon inflation propels the endoscope within the body passage. The sleeve is retained within the dispenser being ready for coupling to the endoscope as soon as it is inserted. The sleeve comprises folded portion 22 and non-inflatable portion 20. The folded portion is retained between a skirt member 50 and a bushing member 52, while the non-inflatable portion is attached to the cap and is intended for covering the bending section of the endoscope when it is inserted within the dispenser. By virtue of this provision inflation of the sleeve does not compromise maneuverability of the bending section during the endoscopic procedure. This arrangement and appropriate coupling mechanism is described in our patent application 60/570,607, whose disclosure is incorporated herein by reference.

The rear housing is provided with a closure portion 54, while the frontal housing is fitted with a detachable shield 56, which purpose will be explained later on. The rear and frontal housing is formed as a hollow body, which interior is sufficient for deployment of additional elements of the assembly as will be explained further. The interior of the rear housing is co-axial with the interior of the frontal housing and together they define a longitudinal transit passage for the insertion tube, when it is inserted in the dispenser and advanced therealong.

Located between the rear housing and the intermediate bushing a plunger member 58 is provided, which is fitted with a flange 60. Configured as a horseshoe a safety catch 62 is put on an outside periphery of the frontal housing provided between the intermediate bushing and the frontal housing. The safety catch is provided with some resiliency, which keeps it in place, however it is manually detachable from the frontal housing.

The plunger member surrounds the rear housing and is longitudinally displaceable along the dispenser inside a skirt portion 64 of the intermediate bushing. This displacement is allowed when the catch is detached from the frontal housing. The plunger member is displaceable between a most retracted position, in which it is located near the closure portion 54, an intermediate position, in which it approaches a shoulder 66 of the intermediate bushing, and a most protracted position, in which the intermediate bushing approaches the frontal housing.

The plunger member is provided with a shoulder 68 and an annular space 70 is provided between this shoulder and the shoulder 66 of the intermediate bushing. This space is filled with appropriate antifriction fluid material (e.g. silicon oil, glyzerin, etc.). The antifriction fluid material enters into the annular space during manufacture of the dispenser via an appropriate inlet (not shown), which is then sealed.

A spongy material 72 lines an inwardly facing cylindrical surface 74 of the rear housing. This spongy material can be, for example, foamed plastic or any other suitable material capable of absorbing and retaining the antifriction fluid. The lining is configured as a tubular body, which interior constitutes a portion of the longitudinal transit passage, through which passes the multilument tubing and through which the insertion tube can be displaced forth and back within the dispenser.

Sealing rings 76,78,80 are provided, which respectively seal between the rear housing and the plunger member, between the plunger member and the intermediate bushing and between the intermediate bushing and the frontal housing.

The forward end of the rear housing is fitted with a distribution ring 82 fitted with a plurality of radial openings 84. These openings are brought into flow communication with the annular space 70 as soon as the plunger member is displaced in its most protracted position.

Deployed within the closure portion 54 a retaining ring 86 and a sealing ring 88 is provided. The retaining ring prevents escape of the antifriction fluid from the transit passage while the sealing ring prevents escape of air from the dispenser, when the sleeve is being inflated during the endoscopic procedure.

Threaded within the opening of the rear closure portion a nut 90 is provided, which holds the assembly in place.

Within the assembly there is provided also a conical ring 92, which has an outwardly facing conical surface 94 snugly fitting with a corresponding inwardly facing conical surface 96 of the frontal housing. The proximal end of the sleeve is anchored between the above conical surfaces and by virtue of this provision it is proximally coupled to the dispenser.

Located within the frontal housing an elongated bushing 98 is provided, which outside diameter is less than the inside diameter of the frontal housing and thus an elongated annular space 100 is provided therebween. This annular space serves for storing a disposable auxiliary sleeve 102. The auxiliary sleeve is anchored to a proximal end of the bushing 98 and can be pulled from the dispenser at the end of the endoscopic procedure so as to protect the endoscope or any other objects or areas outside the patient's body from any contamination, which could reside on the outer surface of the sleeve or within the multilumen tubing.

It is not shown in detail, but should be appreciated that the rear end of the auxiliary sleeve is anchored to the elongated bushing (e.g. by gluing) while its opposite frontal end is coupled to the detachable shield 56. The shield is detachably connected to the bushing, e.g. by an appropriate snapping mechanism (not seen). The shield is provided with wide lateral portions 104,106, which can be conveniently held during detachment from the frontal housing.

Entered within the frontal housing and deployed inside the elongated bushing an alignment member 108 is provided. This member is used during the initial step of the preparation procedure. The alignment member is fitted with an alignment pin 110, which extends along the transit passage. The alignment member is provided with a tail portion 112 for holding by a hand.

The alignment member is located within the dispenser in such a manner that during insertion thereof within the elongated bushing the pin passes through the access opening of the cap and extends along one of the lumens of the multilumen tubing. By virtue of this provision the multilumen tubing is prevented from any undesirable radial displacement during the preparation procedure when the distal part of the endoscope advances through the transit passage. As soon as the optical head reaches the cap and its viewing optics is brought in alignment with the transparent window, the alignment member can be manually removed from the dispenser by holding its tail portion.

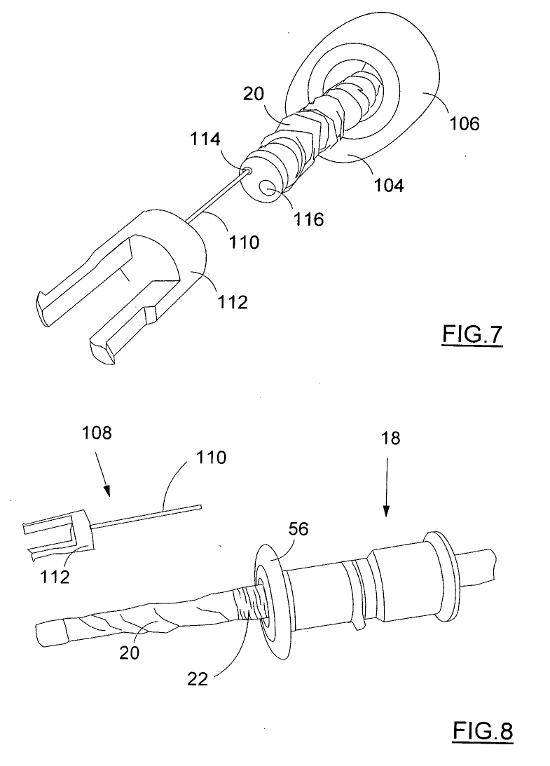

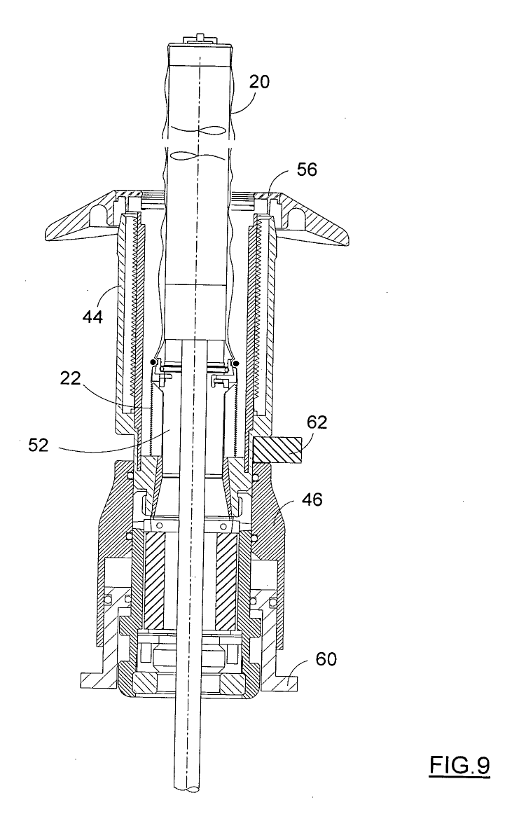

Referring now to FIG. 7 an intermediate step of the preparation procedure is shown. At this step the endoscope is being inserted in the dispenser until its distal end protrudes from the dispenser being covered by the non-inflatable portion 20 of the sleeve. The alignment member is almost removed, while the alignment pin 110 is inserted within an accessing window 114 provided in the cap. It is seen also that a transparent window 116 is provided in the cap, which is brought in alignment with the viewing optics of the camera head. In FIG. 8 and FIG. 9 a disposable dispenser is shown with the distal end of the endoscope protruding outside through the shield 56, while the alignment member is removed. The distal end of the endoscope protrudes from the dispenser and the non-inflatable portion 20 is seen outside, while the folded portion 22 is within the dispenser. It is also seen a portion of the insertion tube at the rear of the dispenser.

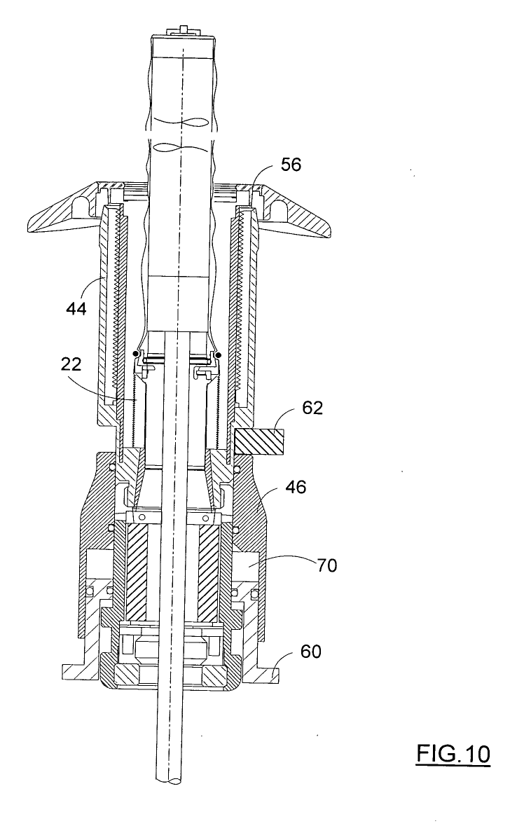

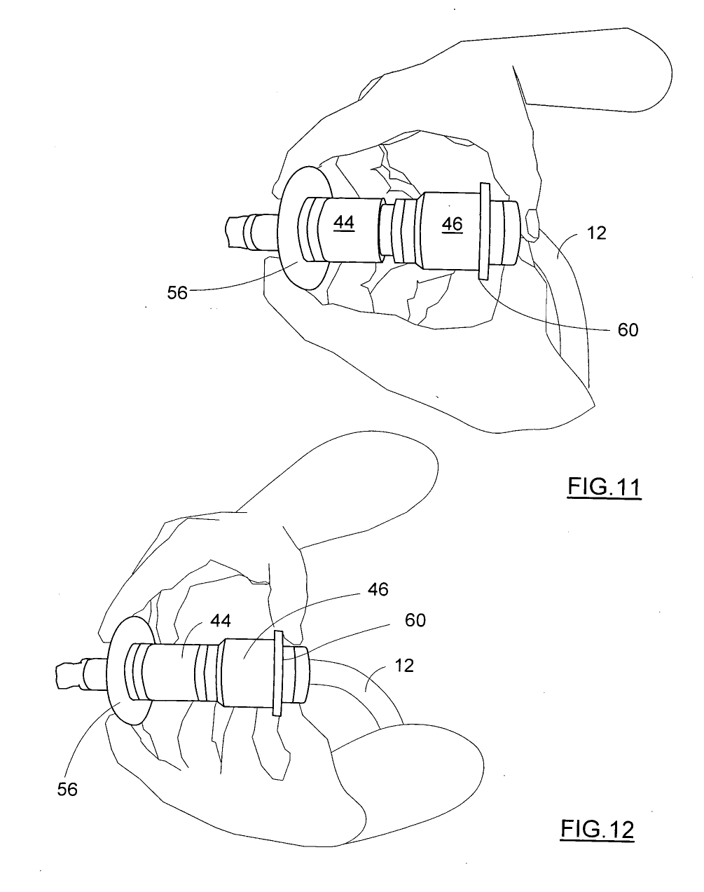

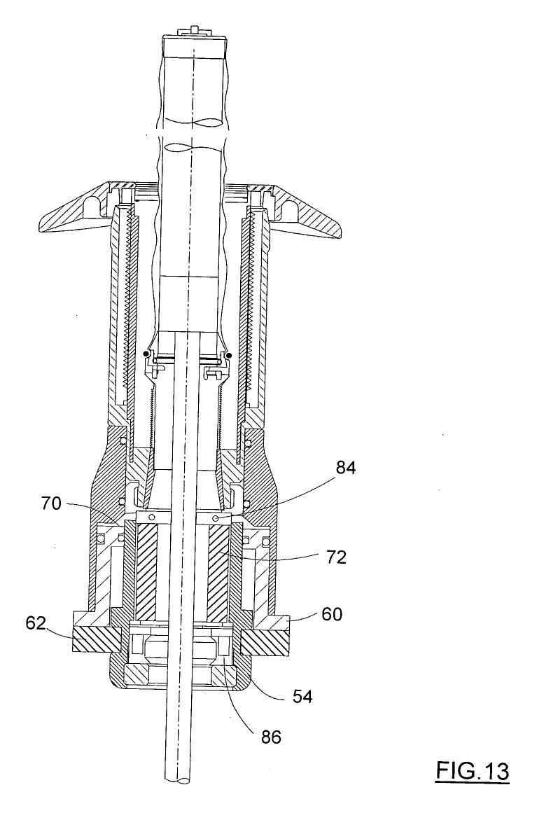

At this point the dispenser is ready for the further step of the preparation procedure. At this step the distal end of the endoscope is to be advanced within the transit passage a bit more until the folded portion 22 is outside the dispenser. This situation is shown in FIG. 10. In this position the dispenser is ready for lubrication, which is carried out by dislodging the antifriction fluid material from the annular space 70 within the transit passage. The antifriction material is distributed within the transit passage so as to prevent or at least to minimize unavoidable friction between the distal end of the endoscope and the transit passage. To carry this out an operator removes safety catch 62 from the frontal housing and holds the dispenser by his/her fingers as shown in FIG. 11. Now, by pushing the flange 60 forward, the operator displaces the plunger member towards the intermediate bushing and then keeps pushing the plunger member further to protract it further together with the intermediate bushing until the intermediate bushing reaches frontal housing 44. This position is depicted in FIGS. 12 and 13. In FIG. 13 is also seen that the safety catch is put on the outside periphery of the rear housing in a position between closure 54 and flange 60. When the plunger member is being pushed forward, the fluid material is dislodged from the annular space 70 towards the distribution ring and then via its openings into the spongy lining 72. The spongy lining absorbs and retains the antifriction material, which lubricates the transit passage. By virtue of this provision, the endoscope moves smoothly along the dispenser. Retaining ring 86 prevents the antifriction material from escaping from the interior of the rear housing.

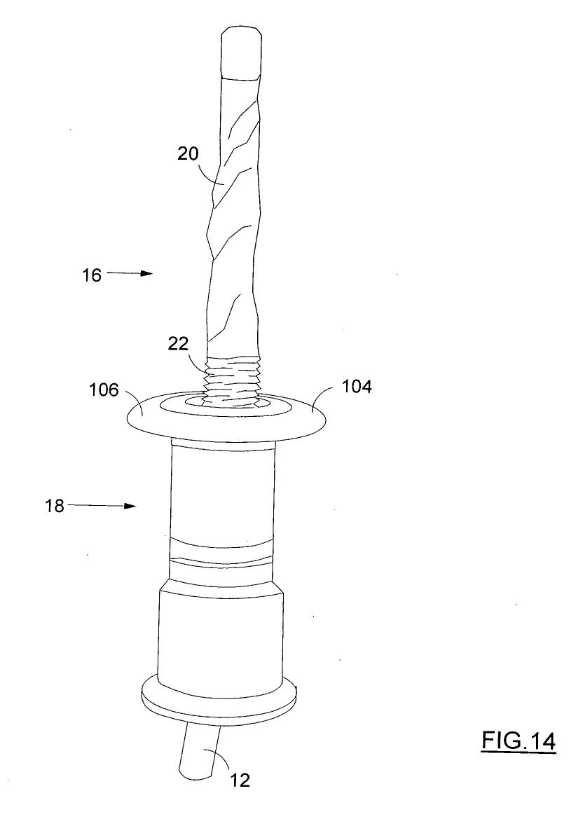

Referring now to FIG. 14 an end step of the preparation procedure is shown. It is seen that the distal section 16 of the endoscope protrudes from the dispenser and is ready for insertion into a body passage. Dispenser 18 and proximal section (not seen) of the endoscope remain outside. During the endoscopic procedure a dedicated garment (not shown) can support lateral portions 104,106 of the frontal shield, for example as explained in Israel patent application 156381. This provision renders the operation of the endoscope more convenient.

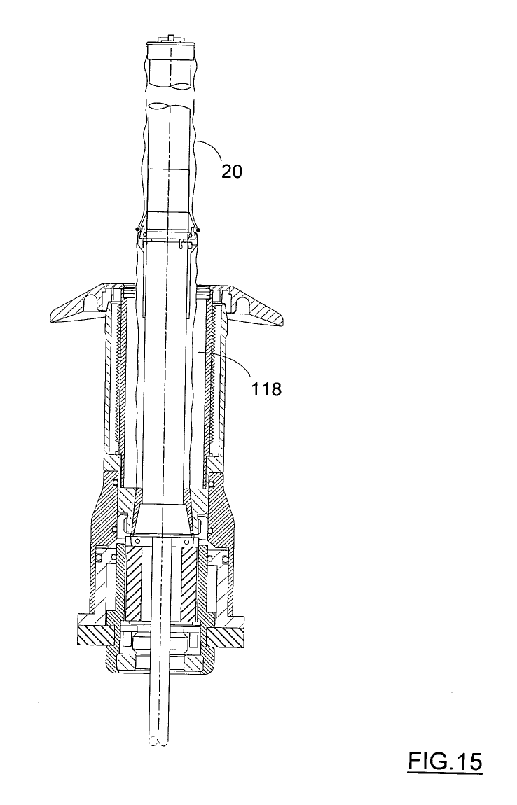

With reference to FIG. 15 an endoscopic apparatus is shown as equipped with the disposable set of the present invention for use during an endoscopic procedure, preferably a colonoscopic procedure. Distal section 16 of the endoscope is covered by a flexible, inflatable sleeve, which upon inflation propels the endoscope within the body passage. The propelling mechanism is based on unfolding and feeding out of the rear folded portion 22 of the sleeve, which becomes an unfolded, tail portion 1118. The tail portion extends proximally and covers the endoscope when it displaces within the body passage. It is not shown specifically, but should be appreciated that the shield and the dispenser always remain outside the body passage.

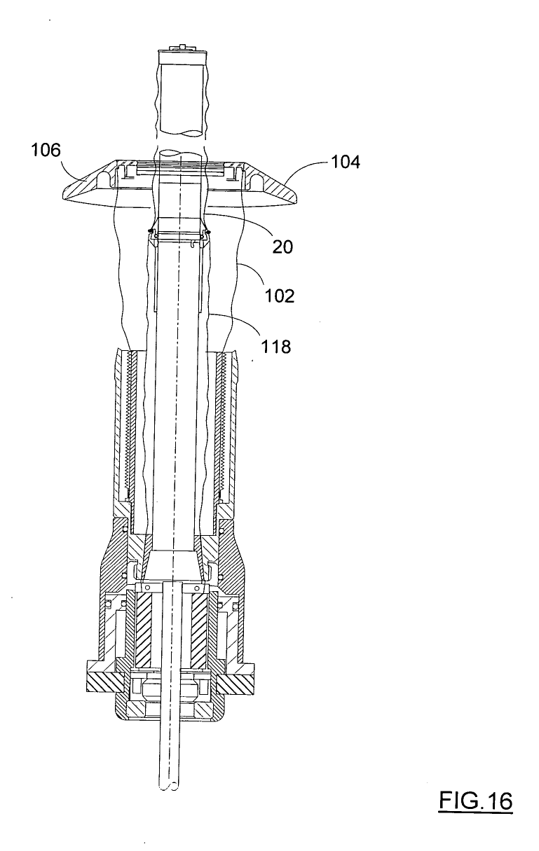

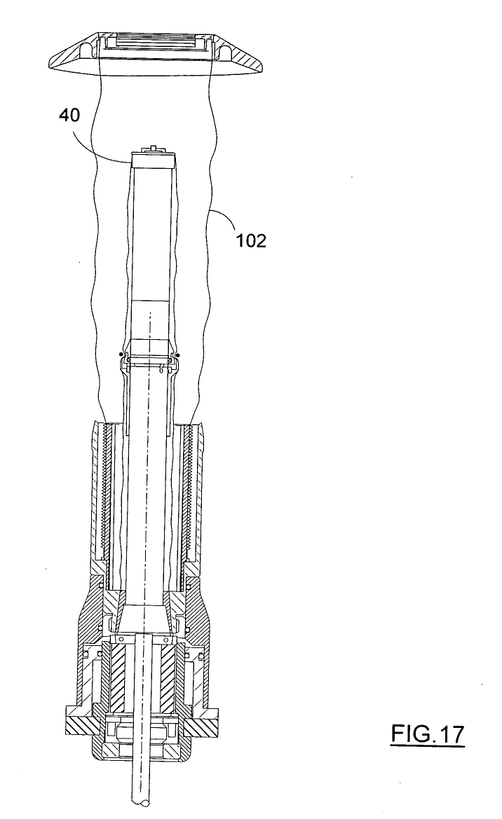

Referring now to FIG. 16 the end of the endoscopic procedure is shown and the endoscope is ready for retracting from the body passage. At this stage the operator detaches the shield from the bushing 98. While retracting the endoscope, the operator pulls the dispenser away from the body passage together with the endoscope. At the same time the operator holds lateral portions 104,106 of the shield and pushes the shield forward towards the patient's body so as to disconnect the shield from the dispenser. As the operator separates the dispenser from the shield, the auxiliary sleeve is gradually pulled out from the frontal housing and covers the flexible sleeve including its non-inflatable portion, remainder of the folded portion and its tail portion. The operator keeps retracting the endoscope until it detaches from the flexible sleeve and optical head 34 detaches from the cap. This situation is shown in FIG. 17. Now the auxiliary sleeve fully covers the flexible sleeve together with the distal portion of the multilumen tubing, thus fully preventing any contamination on the outer surface of the flexible sleeve from contacting the endoscope or any other objects or areas outside the patient's body. After the endoscope has been removed entirely from the flexible sleeve, the auxiliary sleeve is discarded together with the dispenser and the multilumen tubing. It should be born in mind that at the end of the endoscopic procedure the proximal end of the multilumen tubing is sealed to prevent exit of any contamination collected inside its lumens. By virtue of this provision it is possible to prevent the spread of contamination from the flexible sleeve and from the multilumen tubing to the operator's hands or other areas outside the patient's body.

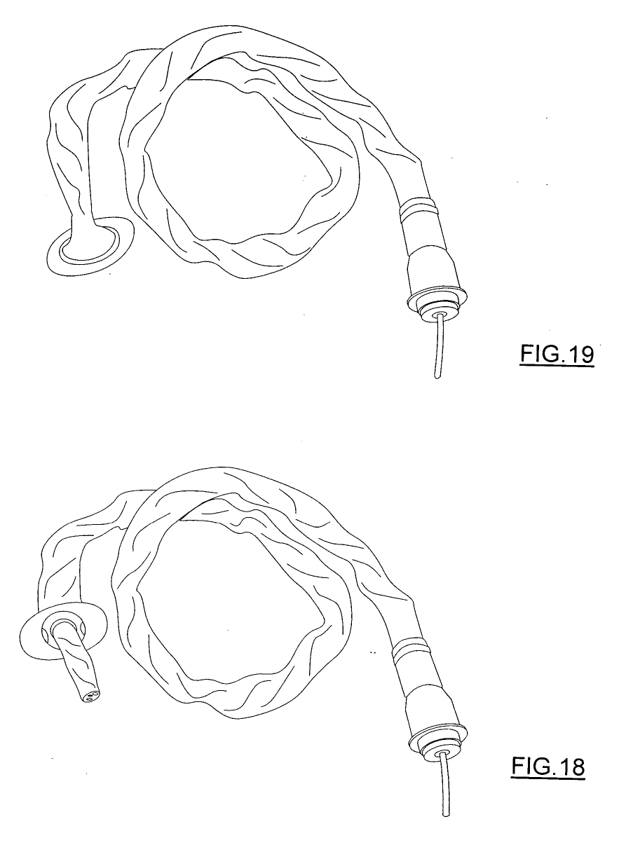

Referring to FIG. 18 it shows when the auxiliary sleeve almost fully covers the flexible sleeve and the distal end of the multilumen tubing. In FIG. 19 it shows when the auxiliary sleeve fully covers the flexible sleeve together with the distal end of the multilumen tubing.

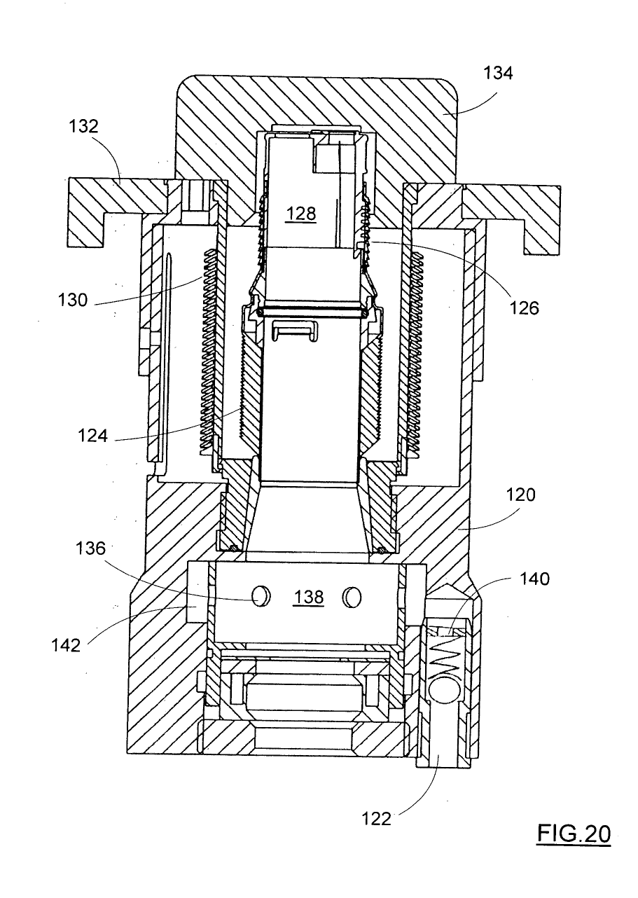

With reference to FIG. 20 an alternative embodiment of the set of the present invention is shown. In accordance with this embodiment for distributing the lubricant in the transit passage, one can employ an external syringe filled with the lubricant. This syringe can be used instead of the dedicated plunger means, which is an integral part of the dispenser.

It is seen that in this embodiment the dispenser is provided with a single housing 120 fitted with a rear port 122. The other components of the set are functionally similar with those of the previous embodiment. In FIG. 20 is seen a protective sleeve having an inflatable, folded portion 124 and a non-inflatable portion 126 attached to a cap 128. It is seen also an auxiliary sleeve 130, stored within a forward portion of the housing and a shield 132 removably attached thereto. A detachable cover 134 closures the frontal portion of the housing and receives therein the cap. For the sake of simplicity the multilumen tubing is not shown. The rear port is in fluid communication with radial openings 136 of a distribution ring 138 via an aperture 140 of a check valve and via an annular space 142. A syringe filled with the lubricant (not shown) would be insertable within the rear port and upon dislodging the lubricant from the syringe, the lubricant would be received in the annular space and then distributed in the transit passage through openings of the distribution ring.

It should be appreciated that the present invention is not limited to the above-described embodiments and that changes and one ordinarily skilled in the art can make modifications without deviation from the scope of the invention, as will be defined in the appended claims.

The set of the present invention can be used with endoscopes, which are not provided with inflatable propelling sleeve.

The set of the invention can be used with flexible or rigid endoscopes.

When used in the following claims, the meaning of terms “comprise”, “include”, “have” and their conjugates is “including but not limited to”.

It should also be appreciated that the features disclosed in the foregoing description, and/or in the following claims, and/or in the accompanying drawings may, both separately and in any combination thereof, be material for realizing the present invention in diverse forms thereof.

1. A disposable set for use with an endoscope wherein said set having a dispenser provided with a longitudinal transit passage for passing therealong of the endoscope, wherein the improvement comprises said dispenser further comprising
a multilumen tubing fitted at a distal end of said dispenser with a cap, said cap being detachably connectable to an optical head of the endoscope; and
a protective sleeve covering the endoscope during an endoscopic procedure, wherein said dispenser is adapted for receiving and distributing a lubricant within the transit passage.

2. The disposable set as defined in claim 1, wherein said dispenser further comprises an annular space for receiving the lubricant thereinto and a distribution ring fitted with radial openings, said openings being in fluid communication with a portion of the transit passage.

3. The disposable set as defined in claim 2, wherein said dispenser further comprises a rear housing, a frontal housing and an intermediate bushing, which is longitudinally displaceable with respect to the frontal housing, wherein the rear housing and the frontal housing being provided with a coaxial interior constituting the transit passage, and an annular space being provided between the rear housing and the intermediate bushing, said annular space being filled with the lubricant and said dispenser is adapted for dislodging the lubricant within the transit passage.

4. The disposable set as defined in claim 3, wherein said rear housing comprises a plunger means longitudinally displaceable along the rear housing to bring the annular space in fluid communication with the radial openings and to dislodge the lubricant therethrough within the transit passage.

5. The disposable set as defined in claim 3, wherein said portion of the transit passage comprises interior of the rear housing.

6. The disposable set as defined in claim 5, wherein said interior being lined with a spongy material capable of absorbing the lubricant.

7. The disposable set as defined in claim 3, wherein said rear housing further comprises a closure portion having a retaining ring and a sealing ring.

8. The disposable set as defined in claim 3, wherein said frontal housing further comprises a bushing member and a skirt member, wherein a proximal portion of the protective sleeve is retained therebetween.

9. The disposable set as defined in claim 6, wherein said frontal housing further comprising an inwardly facing conical surface and a ring having an outwardly facing conical surface snugly fitting with the inwardly facing conical surface, wherein a proximal end of the protective sleeve being anchored between the inwardly facing conical surface and between the outwardly facing conical surface.

10. The disposable set as defined in claim 7, wherein a distal end of the protective sleeve being attached to the cap.

11. The disposable set as defined in claim 8, wherein the proximal portion of the protective sleeve being stored in a bunched state and is inflatable, and wherein a distal portion of the protective sleeve covers a bending section of the endoscope and is non-inflatable.

12. The disposable set as defined in claim 3, wherein said frontal housing further comprising a safety catch preventing inadvertent displacement of the intermediate bushing.

13. The disposable set as defined in claim 1, wherein said cap comprises a window for a viewing optics of the optical head, wherein said set is provided with an alignment member.

14. The disposable set as defined in claim 3, wherein said frontal housing stores therein an auxiliary sleeve pulled from the frontal housing and covering the protective sleeve and the multilumen tubing during their removal from the endoscope.

15. The disposable set as defined in claim 14, wherein an proximal end of the auxiliary sleeve being connected to the frontal housing and a distal end of the auxiliary sleeve being coupled to a shield, which is removably attachable to the frontal housing.

16. A method of preparation of an endoscope for an endoscopic procedure, said method comprising:
a) providing a disposable set consisting of
i) a dispenser fitted with a longitudinal transit passage for passing the endoscope therealong
ii) a multilumen tubing fitted at a distal end thereof with a cap, said cap being detachably connectable to an optical head of the endoscope,
iii) a protective sleeve for covering the endoscope during the endoscopic procedure,
b) inserting a proximal end of the multilumen tubing into a guiding channel of the endoscope,
c) displacing a distal section of the endoscope towards an entry port of the dispenser,
d) inserting the distal section into a dispenser via the entry port,
e) advancing the distal section along the transit passage until the cap is removably attached to the optical head of the endscope, and
e) lubricating the transit passage.

17. The method of preparation as defined in claim 16, comprising aligning of a window provided in the cap with a viewing optics of the optical head.

18. An apparatus for endoscopy, comprising an endoscope for insertion into a body passage during an endoscopic procedure and
a disposable set for use with the enodscope, said set comprises
a dispenser with a longitudinal transit passage for passing the endoscope therealong,
a disposable multilumen tubing fitted at a distal end thereof with a cap detachably connectable to an optical head of the endoscope, and
a protective sleeve covering the endoscope during the endoscopic procedure, wherein said dispenser is adapted for receiving and distributing a lubricant within the transit passage.

19. The apparatus for endoscopy as defined in claim 18, wherein said dispenser comprises an annular space for receiving the lubricant thereinto and a distribution ring fitted with radial openings, said openings being in fluid communication with the transit passage.

20. The apparatus for endoscopy as defined in claim 18, wherein said dispenser comprises a rear housing, a frontal housing and an intermediate bushing, which is longitudinally displaceable with respect to the frontal housing, wherein the rear housing and the frontal housing being provided with a coaxial interior constituting the transit passage, and an annular space being provided between the rear housing and the intermediate bushing, said annular space being filled with the lubricant and said dispenser is adapted for dislodging the lubricant within the transit passage.
